(19) United States
(12) Patent Application Publication
(10) Pub. No.: US 2012/0122087 A1
(43) Pub. Date: May 17, 2012

(54) 5-Hydroxymethylcytosine as a biomarker for early detection, treatment and prognostic monitoring of cancer

(76) Inventors: Weiwei Li, Lindenhurst, NY (US); Jessica Li, Lindenhurst, NY (US)

(21) Appl. No.: 12/927,512

(22) Filed: Nov. 17, 2010

(51) Int. Cl. C12Q 1/68; G01N 33/50

(52) U.S. Cl. 435/6.11; 436/98; 436/94; 435/6.14

(57) ABSTRACT

The present invention is related to the use of 5-hydroxymethylcytosine or a biomolecule having general structure of 5-hydroxymethycytosine as a biomarker for the detection, treatment monitoring, and prognostic prediction of cancer.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

1. Field of the Invention

The present invention is related to the use of 5-hydroxymethylcytosine or a biomolecule having general structure of 5-hydroxymethylcytosine as a biomarker for the detection, treatment monitoring and prognostic prediction of cancer.

2. Description of the Related Art

Detection of cancer at an early stage is critical for successful treatment and increasing survivability. Cancer arises due to the accumulation of DNA alterations that result in cells to uncontrollably proliferate. A common DNA alteration (>95%) is cytosine methylation. Cytosine methylation is an epigenetic modification which is catalyzed by DNA cytosine-5-methyltransferases (DNMTs) and occurs at the 5-position (C5) of the cytosine ring, within CpG dinucleotides. Global 5-methylcytosine (5-mC) in cancer cells is generally reduced compared to that in normal cells. Decrease in global 5-mC or DNA hypomethylation is likely caused by methyl-deficiency due to a variety of environmental influences, and has been proposed as a molecular marker in cancer. It is well demonstrated that the decrease in global 5-mC is one of the most important characteristics of cancer [Feinberg A P et al, Nature, 1983; Gama-Sosa M A et al, Nucleic Acids Res, 1983]. Determination of global 5-mC contents has been used for a biomarker to define diagnostic potential in several types of cancers [Galusca B et al, Virchows Archiv, 2005; Barciszewska, A et al, Therapeutic ribonucleic Acids in Brain Tumor, 2009]. However, the decrease in the level of global 5-mC in cancer cells is not significant compared to that measured in normal cells, which can not allow to sufficiently discriminate cancer states from normal states in a sample tested, as 5-mC content in cancer tissues is only 10-20% lower than that in normal tissues [Finberg A P et al, Cancer Res, 1988; Gama-Sosa M A et al, Nucleic Acid Res, 1983].

Very recently, 5-hydroxymethylcytosine (5-hmC), which is a hydroxylated and methylated form of cytosine has been detected to be abundant in mouse brain and embryonic stem cells [Kriaucionis S et al: Science, 2009]. 5-hmC was first seen in bacteriophages in 1952 (Wyatt G R et al: Nature, 1952) . In mammals, it can be generated by oxidation of 5-mC, a reaction mediated by the Tet family of enzymes [Tahiliani M et al: Science, 2009]. A biomolecule having general structure of 5-hmC may include 5-hydroxymethylcytidine, and 5-hydroxymethyl-2-deoxy-cytidine, and further include 5-hydroxymethyl-2-deoxy-cytidne monophosphate (hmdCMP), 5 -hydroxymethyl-2-deoxy-cytidne diphosphate (hmdCDP), and 5-hydroxymethyl-2-deoxy-cytidne triphosphate (hmdCTP). The broader function of 5-hmC in epigenetics is still unclear. However, a line of evidence showed that 5-hmC plays a role in DNA demethylation, chromatin remodeling and gene expression regulation in a tissue-, cell- or organ-specific manner [Valinluck V et al: Cancer Res, 2007, Valinluck V et al: Nucleic Acid res, 2004, Penn N W et al: Biochem J, 1976, Penn N W et al: biochem J, 1972]. 5-hmC may also negatively regulate cancer formation and development. Numerous evidences showed that methylation-mediated silencing of tumor suppression and apoptosis genes is involved in cancer formation and progression. 5-hmC has been demonstrated to facilitate passive DNA demethylation by blocking the maintenance DNA methyltransferase DNMT1 to methylate DNA containing 5-hmC [Valinluck V et al, Nucleic Acids Res 2004; Liutkeviciute Z et al, Nat Chem Biol 2009] and to participate in active DNA demethylation by enzymatic or spontaneous conversion of 5-mC [Tahiliani M et al Science 2009; Ito S et al, Nature, 2010]. Thus, it is possible that the 5-hmC could affect the reactivation of these genes through 5-hmC-mediated demethylation and help to restore the tumor suppression and apoptosis function of these genes.

### BRIEF SUMMARY OF THE INVENTION

The present invention identifies 5-hmC or a biomolecule having general structure of 5-hmC as a biomarker that can be used in the detection, treatment monitoring and prognostic prediction of cancer.

The present invention also provides a method to measure 5-hmC or a biomolecule having general structure of 5-hmC in a DNA sample of a subject comprising:
(a) obtaining DNA from samples of a subject;
(b) measuring contents of 5-hmC or a biomolecule having general structure of 5-hmC in DNA of a subject;
(c) comparing the measured contents of 5-hmC or contents of a biomolecule having general structure of 5-hmC in DNA of a subject to reference contents of 5-hmC or reference contents of a biomolecule having general structure of 5-hmC measured from a pool of cancer-free individuals, wherein the contents of 5-hmC or contents of a biomolecule having general structure of 5-hmC in DNA of a subject is less than at least 50% of the reference contents, then a subject has cancer or an increased cancer risk.

The present invention also provides a method for health management of a subject. According to the method of this invention, a subject detected to have suspected cancer can be subject to analysis with a traditional diagnostic method such as CT or MRI or colonoscopy, depending on the site, to accurately confirm the presence of a cancer. The present invention also provides a method for monitoring cancer progression of a subject by analyzing body fluid samples. Cancer progression includes therapeutic effectiveness, recurrence and metastasis of a cancer.

### BRIEF DESCRIPTION OF THE DRAWINGS

FIG. 1 shows the general structure of 5-hmC. The R in the structure represents hydrogen group, or deoxyribose, or monophosphate deoxyribose, or diphosphate deoxyribose, or triphosphate deoxyribose.

FIG. 2 shows the diagram of 5-hmC immunodetection.

FIG. 3 shows the contents of 5-hmC measured in different human tissues. The experiment was carried out as described in Example 1.

FIG. 4 shows the contents of 5-hmC measured in different cancer samples. The experiment was carried out as described in Example 2.

### DETAILED DESCRIPTION OF THE INVENTION

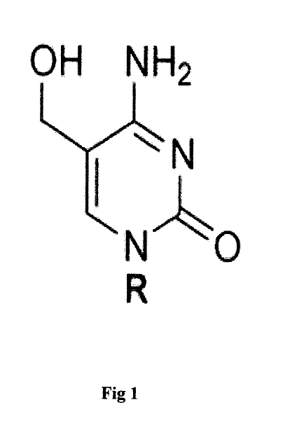

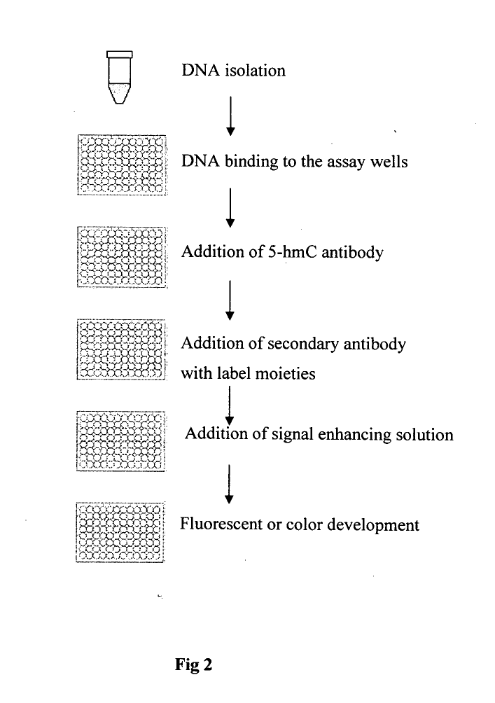

The present invention provides a method for detecting cancer or an increased cancer risk by measuring contents of 5-hmC or contents of the biomolecules having general structure of 5-hmC in a subject. The general structure of 5-hmC is described in FIG. 1 and a basic outline of the method presented in this invention used for measuring contents of 5-hmC or contents of the biomolecules having general structure of 5-hmC is described in FIG. 2. This method is particularly useful for detecting cancer or an increased cancer risk in a subject within a period of short time. This method is also particularly useful for detecting cancer or an increased cancer risk in a subject in a high throughput format. The method of the present invention may also be useful in determining treatment efficacy and recurrence of cancer. Measurement of 5-hmC may be effected following and optionally prior to anti cancer treatment, whereby an increase in 5-hmC or the biomolecules having general structure of 5-hmC in DNA samples is indicative of treatment efficacy, and a decrease in 5-hmC or the biomolecule having general structure of 5-hmC in DNA samples is indicative of recurrence.

According to the method of this invention, the sample for DNA extraction can be from various tissues or body fluids including but are not limited to tissue biopsy, tissue section, formalin fixed paraffin embedded (FFPE) specimens, blood, plasma, serum, bone marrow, cerebro-spinal fluid, tears, sweat, lymph fluid, saliva, nasal swab or nasal aspirate, sputum, bronchoalveolar lavage, breast aspirate, pleural effusion, peritoneal fluid, glandular fluid, amniotic fluid, cervical swab or vaginal fluid, ejaculate, semen, prostate fluid, urine, conjunctival fluid, duodenal juice, pancreatic juice, bile, and stool. DNA could be isolated by lysis of cells with lysis buffer containing a sodium salt, tris-HCl, EDTA, and detergents such as sodium dodecyl sulphate (SDS) or cetyltrimethylammonium bromide (CATB). Tissue fragments should be homogenized before lysing. For example, disaggregating of tissue fragments can be performed by stroking 10-50 times, depending on tissue type, with a Dounce homogenizer. DNA can be further purified by mixing with a high concentration of sodium chloride and then adding into a column pre-inserted with a silica gel, a silica membrane, or a silica filter. The DNA that binds to the silica matrix is washed by adding a washing buffer and eluted with TE buffer or water. DNA can also be isolated and purified by using commercially available DNA extraction kits such as QiaAmp tissue kits. Body fluid should be pre-treated under appropriate condition prior to DNA extraction. For example, if a blood sample is used in this invention, anti-coagulants contained in whole blood should be able to inhibit DNAse activity. A suitable anti-coagulant may be a chelating agent such as EDTA that prevents both DNAse-caused DNA degradation and clotting of the whole blood samples. If other body fluid samples such as sputum are used, Cells in these kinds of samples can be collected by the procedures described in prior art. For example, collection of cells in a urine sample can simply be achieved by simply centrifugation, while collection of cells in a sputum sample requires DTT treatment of sputum followed by filtering through a nylon gauze mesh filter and then centrifugation. If a stool sample is used, a stool stabilizing and homogenizing reagents should be added to stabilize DNA and remove stool particles. Human DNA fraction from total stool DNA then can be primarily isolated or purified using commercially available stool DNA isolation kits such as Qiagen DNA Stool Mini Kit (using the protocol for human DNA extraction) or be captured by methyl-binding domain (MBD)-based methylated DNA capture methods after total DNA isolation [Zhou H et al, Clinical Chemistry, 2007].

Purified DNA can be then used for the measurement of 5-hmC or the biomolecules having general structure of 5-hmC. A person of ordinarily skill in the art will be able to determine the content of 5-hmC or the content of the biomolecules having general structure of 5-hmC using the methods of the present invention by comparing the content of 5-hmC or the content of the biomolecules having general structure of 5-hmC from diseased subjects with reference content measured from healthy or cancer-free individuals.

5-hmC or the biomolecules having general structure of 5-hmC can be determined by many methods well known in the art. A variety of chromatography-based technologies including, without limitation, capillary electrophoresis (CE), mass spectrometry (MS) HPLC and thin layer chromatography (TLC) [Kriaucionis S et al, Science 2009; Penn N W et al, Biochem J 1972; Szwagierczak A et al, Nucleic Acids Res 2010] can be used for measuring 5-hmC or the biomolecules having general structure of 5-hmC.

Preferably immunoassay techniques including competitive and non-competitive immunoassays can be used. A variety of immunoassay techniques include, but are not limited to, enzyme immunoassays (EIA) such as enzyme multiplied immunoassay technique (EMIT), enzyme-linked immunosorbent assay (ELISA), enzyme-linked immunosorbent spot (ELISPOT), microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with time-resolved fluorescence (TRF) assay such as DEFLIA assays; luminescent oxygen channeling assay (LOCI) such as AlphaLISA or AlphaScreen assays; laser induced fluorescence; liposome immunoassays; and immunosensors. Other immunoassay methods such as dot blot immunoassay, immunohistochemical staining, and immunofluorescence assays can be also used. Immunoassay methods and protocols are generally described in the prior art.

More preferably, an immunoassay method specifically developed for quantification of 5-hmC can be used. This method has been developed by the inventors (U.S. patent application Ser. No. 12/803,666). The use of this method is able to allow 5-hmC quantification to be much easier and more convenient than existing methods, as the method can be carried out with common equipment such as a microplate reader or microscope, and can be performed in a high throughput format with high sensitivity and specificity.

According to the invention, antibodies against 5-hmC or the biomolecules having general structure of 5-hmC are commercially available [Active Motif, Abgent and Diagenode]. Antibodies can also be produced using various techniques known in the art, including phage display libraries [Hoogenboom et al, J. Mol. Biol., 1991; Marks et al., J. Mol. Biol., 1991]. Monoclonal antibodies can be generated using the techniques of Cole et al. and Boerner et al [Cole et al., Monoclonal Antibodies and Cancer Therapy, 1985; Boerner et al., J. Immunol., 1991]. Polyclonal antibodies can be generated by using the Abgent protocol: (1) preparation of 5-hmC—KLH conjugates. KLH may be modified with 3-sulfo-N-hydroxysuccinimide ester sodium salt before conjugation. The conjugates of KLH-5-hmC can be identified by ultraviolet spectrophotometry. (2) injection of KLH-5hMC into rabbits. Injections of the antigen are given in multiple sites to stimulate the best immunity. The rabbits are boosted at 21 day intervals until peak antibody titers are reached (6-8 re-immunizations); (3) blood sample collection. Blood is collected from the central ear artery and allowed to clot and retract at 37° C. overnight. The clotted blood is then refrigerated for 24 hours before the serum is decanted and clarified by centrifugation; and (4) ELISA test of antibody titers and affinity purification.

DNA hydroxymethylated at every cytosine can be used as an internal positive control. To prepare the internal positive control, PCR amplicons with a length of 357 bp were generated using human MLH1 promoter derived sequences by incorporating dhmCTP (5-hydroxy-methylcytidine) with dATP, dGTP, and dTTP. DNA methylated at every cytosine is used as the comparative control. To prepare the comparative control, PCR amplicons with a length of 357 bp were generated using human MLH1 promoter derived sequences by incorporating dmCTP (5-methylcytidine) with dATP, dGTP, and dTTP. DNA unmethylated at every cytosine (unC) is used as the internal negative control. To prepare the internal negative control, PCR amplicon with, length of 357 bp were generated using human MLH1 promoter derived sequences by incorporating dCTP (cytidine) with dATP, dGTP, and dTTP. The positive control, comparative control and negative control contain 25% of 5-hmC, 5-mC and unC, respectively. Each control with a known amount of 5-hmC, 5-mC, and unC can be used concomitantly with DNA samples of a subject.

A variety of detectable moieties can be used in the assays described herein. The appropriate label moieties can be chosen based on the sensitivity required, ease of conjugation with the antibody, stability requirements, and available instrumentation and disposal provisions. Suitable detectable moieties include, without limitation, horse radish peroxidase (HRP), alkaline phosphotase (AP), biotin, fluorescein (FITC), Cy3, Cy5, rhodamine, dynabeads, texas red, Alexa fluor, BODIPY, captivate ferrofluid, cascade blue, beta-lactamase, marine blue, nanogold, Oregon green, pacific blue, radionuclides quantum dot, green fluorescent protein (GFP), phycoerythrin and the like.

The detectable moieties can be labeled to the antibody used for specific binding to an epitope. Direct labels include fluorescent or luminescent tags, metals, dyes, radionuclides, and the like, which are attached to the antibody. Direct labels include, but are not limited to, fluorescein, Cy3, Cy5, rhodamine, dynabeads, texas red, Alexa fluor, BODIPY, R-phycocyanin, B-phycoerythrin, R-phycoerythrin, rhodamine, nanoglod, and quantum dot. Indirect labels include a variety of enzymes well known in the art, such as horseradish peroxidase (HRP), alkaline phosphatase (AP), biotin, beta-lactamase, and the like. For using indirect label detection, a chromogenic substrate should be used. For example, using tetramethylbenzidine (TMB) for an HRP labeling system will generate a soluble color product in the presence of hydrogen peroxide, which is detectable at absorbance wavelength of 450 nm. Using chromogenic substrate p-nitrophenyl for alkaline phosphatase detection system gives a soluble product detectable at 405 nm.

For some of immunoassays such as sandwich ELISA or ELISPOT, the unlabeled antibodies can be immobilized onto a variety of solid carriers, which include but are not limited to polystyrene beads, magnetic or chromatographic matrix particles, microtiter wells, plastic or nylon membranes, plastic or glass slides, and the like. For some of immunoassays such as direct ELISA, the DNA isolated from a sample can be directly bound onto a variety of solid carriers.

A fluorescent signal from the direct labels can be read with a fluorescent detection instrument in the presence of appropriate light at a defined wavelength. Fluorescent detection instruments such as a Flx800 fluorescence microplate reader [Biotek] are commercially available. A colorimetric signal from the indirect labels can be read with an absorbance detection instrument such as a Max-kinetic microplate reader [Molecular Devices, CA] with the appropriate wavelength. The assays can also be automated or performed robotically based on the requirement, and the signal from multiple samples can be detected simultaneously.

It is unexpected that in the assays based on the methods of this invention, the content of 5-hmC was found to be greatly varied in different human tissues, which indicates that 5-hmC distribution is tissue-specific in human. It is also unexpected that in some types of tissues, the content of 5-hmC is significantly lower in cancer states than that in normal states. The difference of 5-hmc content between cancerous tissues and normal tissues from health subjects is significantly sufficient to distinguish cancer states from normal states. Further, it has been discovered that the use of the immunoassay method specifically for 5-hmC quantification is able to measure content of 5-hmC with high specificity and sensitivity in a high throughput format.

The method of this invention for early cancer detection is further illustrated in the following examples:

### EXAMPLE 1

The experiment was carried out to measure the contents of 5-hmC in different human tissues.

DNA was isolated from frozen tissues of human brain, lung, heart, breast, liver, kidney, uterus, colon-rectum, placenta, and from peripheral blood lymphocytes (PBL). The concentrations of all DNA samples are measured with a spectrophotometer and confirmed with a PicoGreen dsDNA quantitation kit (Invitrogen CA). A 260/280 ratio of all DNA samples is greater than 1.8.

DNA fragments containing cytosine, 5-mC or 5-hmC were amplified by PCR using a region of hMLH1 containing promoter and exon1. A starting amount of 1 ng of human placenta DNA was used to generate 693 bp DNA amplicons by PCR reactions with a reaction buffer containing 0.2 mM of each dNTP (or 5mdCTP or 5hmdCTP in place of dCTP), and Phire hot start polymerase [Finnzymes, MA]. PCR reactions in 20 μl reaction volumes were carried out according to the manufacturer's instructions. To effectively remove unmodified DNA templates from the final products, subsequent PCR amplifications were performed using 1 μl of first round PCR products in 20 μl of reaction volume under the same reaction conditions with different primers to generate 357 bp DNA products. These DNA products contain about 25% cytosine, 25% 5-mC or 25% 5-hmC, respectively. PCR products were then run on a 1.5% agarose gel to confirm the correct length and were purified by a sodium acetate/ethanol precipitation method.

To measure 5-hmC content, 200 ng of DNA were immobilized onto assay wells of microplates. The wells were washed 3 times with PBS-T and 5-hmC polyclonal antibody [Active Motif, CA] was added into the wells at 1 μg/ml and incubate at room temperature for 60 min. After washing 3 times, biotin-conjugated anti-rabbit antibody [Fisher, IL] at 0.2 μg/ml was added and incubated at room temperature for 30 min. After washing with PBS-T 4 times, the signal enhancing solution containing avidin-proxidase complex was added and incubated for 30 min. After washing with PBS-T 4 times, TMB (100 μl per well) was added and incubated at room temperature for 10 min, and then 50 μl per well of 1M HCl was added to stop the enzymatic reaction. The reference DNA fragments containing 5-hmC and 5-mC were used as the positive standard and negative control, respectively. The absorbance end point (optical density, OD) was read on a Max-kinetic microplate reader (Molecular Devices, CA). The amount of 5-hmC is proportional to the OD intensity measured. After subtracting negative control readings from the readings for the sample and the standard, the value of 5-hydroxymethylcytosine for each sample was calculated as a ratio of sample OD relative to the OD of the standard.

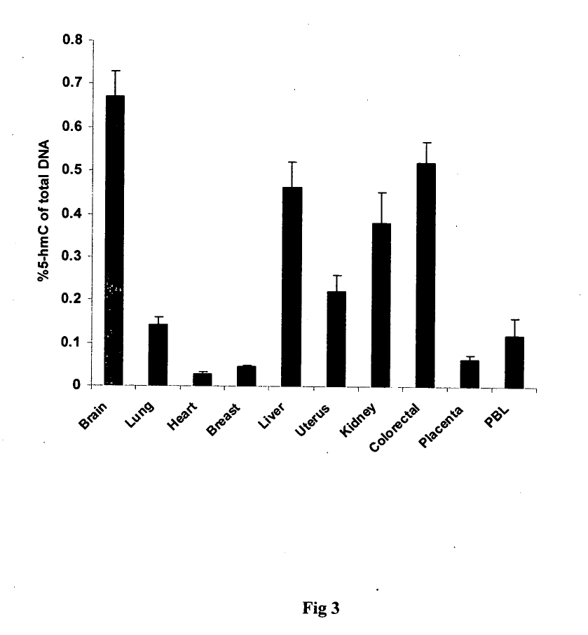

It was also observed that 5-hmC was abundant in brain (0.67%), kidney (0.38%), colorectal (0.52%), liver (0.46%), and uterus (0.22%) tissues. In contrast, 5-hmC content was relatively low in lung (0.14%) and PBL (0.12%), and very low in heart (0.03%), breast (0.05%) and placenta (0.06%).The results are shown in FIG. 3.

### EXAMPLE 2

The experiment was carried out to measure the contents of 5-hmC in cancerous tissues.

DNA was isolated from frozen tissues of 3 colorectal cancer, 2 kidney cancer, 2 cervical cancer, 2 colon cancer cell lines HCT116 and SW620, and cervical cancer cell line He1a, The concentrations of all DNA samples are measured with a spectrophotometer and confirmed with a PicoGreen dsDNA quantitation kit (Invitrogen CA). A 260/280 ratio of all DNA samples is greater than 1.8.

To measure 5-hmC content, 200 ng of DNA were immobilized onto the assay wells of microplates. The wells were washed 3 times with PBS-T and 5-hmC polyclonal antibody [Active Motif, CA] was added into the wells at 1 μg/ml and incubated at room temperature for 60 min. After washing 3 times, biotin-conjugated anti-rabbit antibody [Fisher, IL] at 0.2 μg/ml was added and incubate at room temperature for 30 min. After washing with PBS-T 4 times, the signal enhancing solution containing avidin-proxidase complex was added and incubated for 30 min. After washing with PBS-T 4 times, TMB (100 μl per well) was added and incubated at room temperature for 10 min, and then 50 μl per well of 1M HCl was added to stop the enzymatic reaction. The reference DNA fragments containing 5-hmC and 5-mC were used as the positive standard and negative control, respectively. The absorbance end point (optical density, OD) was read on a Max-kinetic microplate reader (Molecular Devices, CA). The amount of 5-hmC is proportional to the OD intensity measured. After subtracting negative control readings from the readings for the sample and the standard, the value of 5-hydroxymethylcytosine for each sample was calculated as a ratio of sample OD relative to the OD of the standard.

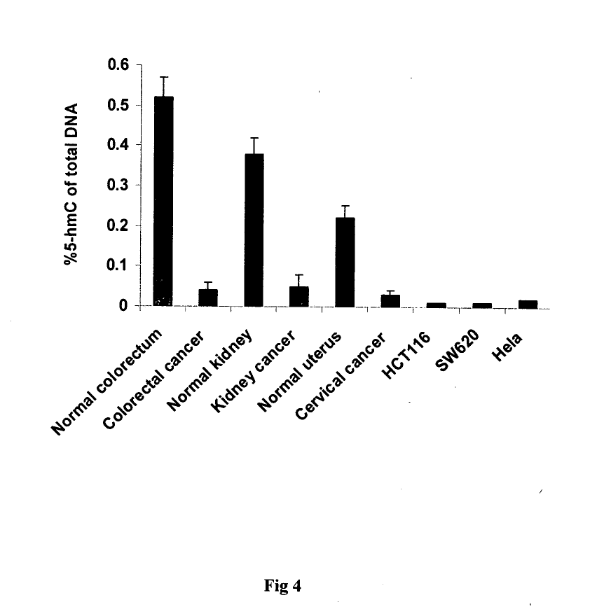

It was also observed that the content of 5-hmC in cancerous tissues and cancer cell lines is much lower than that in the same type of normal tissues. The results are shown in FIG. 4 and Table 1.

TABLE 1
Contents of 5-hmC in human normal and cancer tissues

| | %5-hmC of total DNA | | |
|---|---|---|---|
| Tissue | normal states | cancerous states | Cancer/normal ratio |
| Colorectal | 0.52 | 0.04 | 7.7% |
| Kidney | 0.38 | 0.05 | 13% |
| Uterus | 0.22 | 0.03 | 14% |

What we claim is:

1. A method for detecting cancer or an increased cancer risk by measuring contents of 5-hydroxymethylcytosine or contents of a biomolecule having general structure of 5-hydroxymethylcytosine in a subject comprising: (a) obtaining DNA from samples of said subject; (b) measuring contents of said 5-hydroxymethylcytosine or contents of said biomolecule having general structure of 5-hydroxymethylcytosine in said DNA of said subject; and (c) comparing the measured contents of said 5-hydroxymethylcytosine or contents of said biomolecule having general structure of 5-hydroxymethylcytosine in said DNA of said subject to reference contents of said 5-hydroxymethylcytosine or contents of said biomolecule having general structure of 5-hydroxymethylcytosine, which is measured from a pool of cancer-free individuals, wherein the contents of said 5-hydroxymethylcytosine or contents of said biomolecule having general structure of 5-hydroxymethylcytosine in said DNA of said subject is less than at least 50% of said reference contents, then said subject has cancer or an increased cancer risk.

2. The method according to claim 1 wherein said samples are tissue biopsy, tissue section, formalin fixed paraffin embedded (FFPE) specimens, blood, plasma, serum, cerebro-spinal fluid, tears, sweat, lymph fluid, bone marrow, saliva, nasal swab or nasal aspirate, sputum, bronchoalveolar lavage, breast aspirate, pleural effusion, peritoneal fluid, glandular fluid, amniotic fluid, cervical swab or vaginal fluid, ejaculate, semen, prostate fluid, urine, conjunctival fluid, duodenal juice, pancreatic juice, bile, stool, or any combination thereof.

3. The method according to claim 1 wherein said biomolecule having general structure of 5-hydroxymethylcytosine is 5-hydroxymethylcytosine, 5-hydroxymethylcytidine, 5-hydroxymethyl-2-deoxy-cytidine, 5-hydroxymethyl-2-deoxy-cytidine monophosphate, 5-hydroxymethyl-2-deoxy-cytidne diphosphate, or 5-hydroxymethyl-2-deoxy-cytidne triphosphate.

4. The method according to claim 1 wherein said cancer is a solid tumor or hematological tumor.

5. The method according to claim 1 wherein said cancer is leukemia, colorectal cancer, liver cancer, kidney cancer, lung cancer, cervical cancer and brain cancer.

6. The method according to claim 1 wherein said cancer is cervical cancer, colorectal cancer, and kidney cancer.

7. The method according to claim 1 wherein the contents of said 5-hydroxymethylcytosine or contents of said biomolecule having general structure of 5-hydroxymethylcytosine are measured by using a technique selected from the group comprising HPLC, TLC, electrochemical analysis, capillary electrophoresis, mass spectrometry, fluorescent analysis, gas chromatography (GC), radiochemical analysis, or immunological analysis.

8. The method according to claim 1 wherein the contents of said 5-hydroxymethylcytosine or contents of said biomolecule having general structure of 5-hydroxymethylcytosine are measured by using immunological analysis comprising ELISA, dot blot, immunohistochemistry and immunofluorescence.

9. The method according to claim 1 wherein the contents of said 5-hydroxymethylcytosine or contents of said biomolecule having general structure of 5-hydroxymethylcytosine are measured by using immunological analysis comprising direct ELISA, sandwich ELISA, and competitive ELISA.

10. The method according to claim 1 wherein the contents of said 5-hydroxymethylcytosine or contents of said biomolecule having general structure of 5-hydroxymethylcytosine are measured in multi-well plate, microchip, microscope slide, and nitrocellulose membrane.
